(19) United States
(12) Patent Application Publication
(10) Pub. No.: US 2019/0021635 A1
(43) Pub. Date: Jan. 24, 2019

(54) EARLY STROKE DETECTION DEVICE

(71) Applicant: George P. Teitelbaum, Santa Monica, CA (US)

(72) Inventor: George P. Teitelbaum, Santa Monica, CA (US)

(21) Appl. No.: 16/068,828

(22) PCT Filed: Jan. 13, 2017
(86) PCT No.: PCT/US2017/013497

§ 371 Date: Jul. 9, 2018

Related U.S. Application Data
(60) Provisional application No. 62/278,740, filed on Jan. 14, 2016.

(51) Int. Cl. A61B 5/1459; A61B 5/1455; A61B 5/00; A61B 1/07

(52) U.S. Cl. CPC A61B 5/1459; A61B 5/14552; A61B 5/6852; A61B 1/07; A61B 2562/0238; A61B 5/4064; A61B 5/7282; A61B 2560/0219; A61B 5/0013

(57) ABSTRACT

A stroke detection device (50, 100), and associated methods of operation, for early detection of ischemic stroke. The device (50, 100) includes a fiberoptic port (20, 102) connected to an end of a fiberoptic catheter (22, 120), the catheter (22, 120) including a first optical fiber (26, 122) and a second optical fiber (28, 124) each extending along at least a portion of the catheter (22, 120). The catheter (22, 120) is configured to direct infrared light along the first optical fiber to illuminate a subcutaneous region of the patient, and to further obtain reflected light data via the second optical fiber based on the infrared light reflected from cells present in the subcutaneous region. Based on the reflected light data, the stroke detection device (50, 100) monitors SjVO2 levels for early detection of ischemic strokes.

### TECHNICAL FIELD

The field of the present disclosure relates generally to medical devices, and in particular, to such medical devices operable to detect a stroke in its early stages.

### BACKGROUND

A stroke occurs when the blood supply to a person's brain is interrupted or severely reduced, thereby depriving brain tissue of oxygen and nutrients. Strokes can be classified into two major categories: ischemic and hemorrhagic. Ischemic strokes, which account for approximately 83% of strokes, are caused by interruption of the blood supply to the brain, such as when a blood clot or other debris blocks a blood vessel in the brain or one leading to it. Hemorrhagic strokes typically occur when a blood vessel ruptures in the brain. The resulting bleeding deprives downstream brain cells of oxygenated blood and can also damage cells by increasing pressure inside the brain. Early detection of ischemic strokes, especially those occurring during sleep, is more difficult than detection for hemorrhagic strokes since ischemic strokes generally occur without pain. However, as demonstrated in the scientific literature, early detection and treatment of ischemic strokes is significantly more effective.

To this end, the medical community has developed a number of different devices for early detection of ischemic strokes. For example, one device includes a wearable headpiece operable to track brainwaves and analyze a number of neurological health markers to alert the user of the very earliest signs of an impending stroke. Another device includes a wearable wrist watch designed to detect circulating blood clots using photoacoustic flow cytometry. Still another device uses ultrasound technology for identifying arterial plaque that is at high risk of breaking off and causing heart attack or stroke.

Each of these devices has certain disadvantages, such as high cost and/or require equipment that is worn by the user. The following disclosure relates to an implantable stroke detection device operable to provide reliable early detection of strokes. Additional aspects and advantages of such improved stroke detection devices may be apparent from the following detailed description of example embodiments, which proceeds with reference to the accompanying drawings.

Understanding that the drawings depict only certain embodiments and are not, therefore, to be considered limiting in nature, embodiments relating to a stroke detection device will be described and explained with additional specificity and detail with reference to the drawings.

### BRIEF DESCRIPTION OF THE DRAWINGS

FIG. 1 is a schematic view illustrating a fiberoptic port of a stroke detection device implanted in the internal jugular vein of a patient in accordance with one embodiment.

FIG. 2 is a schematic view illustrating the implanted fiberoptic port of FIG. 1 within the subcutaneous tissue of the patient.

FIG. 3 is a schematic view illustrating a transmitter/sensor device of the stroke detection device attached along the exterior skin of the patient to communicate with the implanted fiberoptic port.

FIG. 4 is a schematic view illustrating another embodiment of a stroke detection device.

FIG. 5 is a schematic drawing illustrating internal electronics and components of the stroke detection device of FIG. 4.

### DETAILED DESCRIPTION OF EXAMPLE EMBODIMENTS

With reference to the drawings, this section describes particular embodiments and their detailed construction and operation. The embodiments described herein are set forth by way of illustration only and not limitation. The described features, structures, characteristics, and methods of operation may be combined in any suitable manner in one or more embodiments. In view of the disclosure herein, those skilled in the art will recognize that the various embodiments can be practiced without one or more of the specific details or with other methods, components, materials, or the like. In other instances, well-known structures, materials, or methods of operation are not shown or not described in detail to avoid obscuring more pertinent aspects of the embodiments.

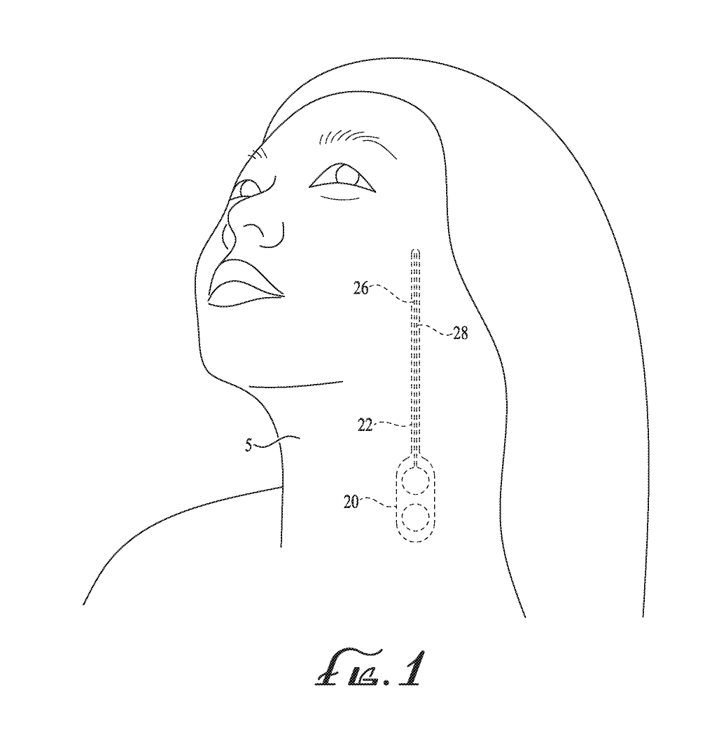

FIGS. 1-5 and the associated discussion below describe various embodiments of an implantable stroke detection device 50, 100 operable to analyze jugular bulb venous blood oxygen saturation (SⱼVO₂) levels for early detection of a stroke. Briefly, jugular bulb venous oxygen saturation (SjvO₂) is the percentage of oxygen bound to hemoglobin in the blood returning to the heart from the brain via the internal jugular vein. In healthy individuals, SjVO₂ levels typically range between 55% and 71%. When cerebral blood flow drops due to an acute ischemic stroke, but there is no concomitant drop in cerebral metabolism, there is usually an increased oxygen extraction of the decreased volume of arterial blood entering the brain. This increased oxygen extraction, in turn, results in decreased SⱼVO₂ levels for the blood exiting the brain. Accordingly, by monitoring the SⱼVO₂ levels in the blood exiting the brain (i.e., by monitoring whether SⱼVO₂ levels are steadily decreasing), the stroke detection device can detect early signs of an acute ischemic stroke. The following describes additional details of the stroke detection devices 50, 100 and their methods of operation.

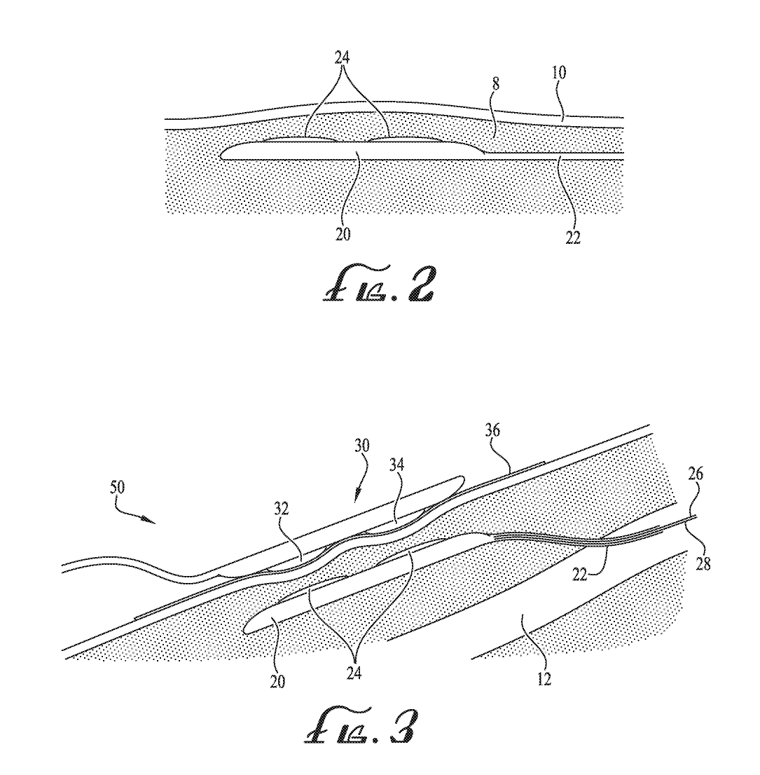

FIGS. 1 and 2 collectively illustrate various components of a stroke detection device 50 operable to use spectrophotometric analysis of the SⱼVO₂ levels in the patient's 5 blood for early detection of strokes. With reference to FIGS. 1 and 2, the stroke detection device 50 includes a fiberoptic port 20 connected to a fiberoptic catheter 22, where the device 50 is implantable into a subcutaneous tissue pouch 8 of a patient 5. The fiberoptic port 20 is a substantially flat and thin light receiving and emitting port, having approximate dimensions of 2 cm×3 cm and a thickness of approximately 5 mm. The fiberoptic port 20 includes one or more light receivers/emitters 24 disposed on an upper surface of the port 20, the receivers/emitters 24 facing the patient's skin 10 as illustrated in FIG. 2, and generally arranged to communicate with a transmitter/sensor device 30 (see FIG. 3). One end of the port 20 is connected to a fiberoptic catheter 22. The catheter 22 may be any suitably-sized catheter, such as a 3F (outer diameter) catheter 22, that contains two or more optical fibers 26, 28 running the length of the catheter 22. To accommodate the various lengths of the internal jugular vein 12 found in the population, the catheter 22 and optical fibers 26, 28 may be provided in various lengths, such as approximately 10-12 cm.

In an example insertion procedure, the catheter 22 is advanced via the internal jugular vein 12 until the fiberoptic port 20 is positioned in the subcutaneous tissue pouch 8 approximately less than 1 cm below the surface of the patient's skin 10. The distal tip (not shown) of the catheter 22 may be in the jugular bulb at the skull base of the patient 5. In some embodiments, the shaft of the catheter 22 may be coated with a lubricious or hydrophilic coating to prevent blood clot and/or fibrous accumulation when implanted. Because the port 20 and catheter 22 are mostly enclosed under the skin, the risk of infection is greatly reduced. In addition to reducing the risk of infection, isolating the port 20 under the skin makes the stroke detection device 40 more convenient and cosmetically appealing for active, ambulatory patients 5. As is discussed in further detail below, the fiberoptic port 20 and catheter 22 transmit and receive light from the catheter 22 through the patient's skin for the spectrophotometric analysis.

With reference to FIG. 3, the stroke detection device 50 further includes an external transmitter/sensor device 30. The transmitter/sensor device 30 includes a light source 32 operable to generate infrared light, and a light reception/sensor 34 operable to detect/receive reflected light. In an example operation process, the transmitter/sensor device 30 is aligned with the fiberoptic port 20 at the proximal end of the catheter 22. In some embodiments, an adhesive pad 36 may be used to hold the device 30 firmly against the skin 10 overlying the fiberoptic port 20 during use.

Once the device 30 is aligned with the port 20, the light source 32 is activated and transmits infrared light to the receivers 24 of the port 20. The fiberoptic catheter 24 directs the infrared light along one of the fiberoptic fibers 26 to the distal tip of the intravenous catheter 24, thereby illuminating the nearby subcutaneous region. As light reflects from the red blood cells, the second optical fiber 28 detects the reflected light in the region and transmits the detected reflected light through the skin 10 and to the photodetector/sensor device 30. The device 30 thereafter analyzes the data or transmits the data to an external computer system for analysis.

Using reflective spectrophotometric analysis, the reflected light data is analyzed to determine the relative quantity of oxyhemoglobin and deoxyhemoglobin in the patient's blood. When the oxyhemoglobin-to-deoxyhemoglobin ratio increases (i.e., increasing SⱼVO₂ levels), red blood cells change in color from purple to scarlet. Conversely, when the oxyhemoglobin-to-deoxyhemoglobin ratio decreases (i.e., decreasing SⱼVO₂ levels), red blood cells change in color from scarlet to purple. Accordingly, by monitoring the color data (e.g., scarlet and purple) based on the reflected light levels, the photodetector/sensor device 30 may be used to determine whether the oxyhemoglobin-to-deoxyhemoglobin ratio is increasing or decreasing.

In this manner, the implanted stroke detection device 50 may be able to provide spectrophotometric analysis of the SⱼVO₂ bilaterally, where an acute unilateral or bilateral drop in jugular bulb SⱼVO₂ levels (especially if it drops below 55%) may indicate an acute significant drop in arterial blood supply to the brain as seen in ischemic stroke. This would be especially useful for detecting stroke during sleep, particularly, in high-risk patients, such as those suffering from atrial fibrillation. Once a sustained drop in SⱼVO₂ levels are detected using the fiberoptic catheter 22, the patient may be awakened by an alarm (and/or another individual may be alerted) so that the patient could be checked (either by a family member, medical personnel, or other caretaker) to determine whether the patient may be having a stroke.

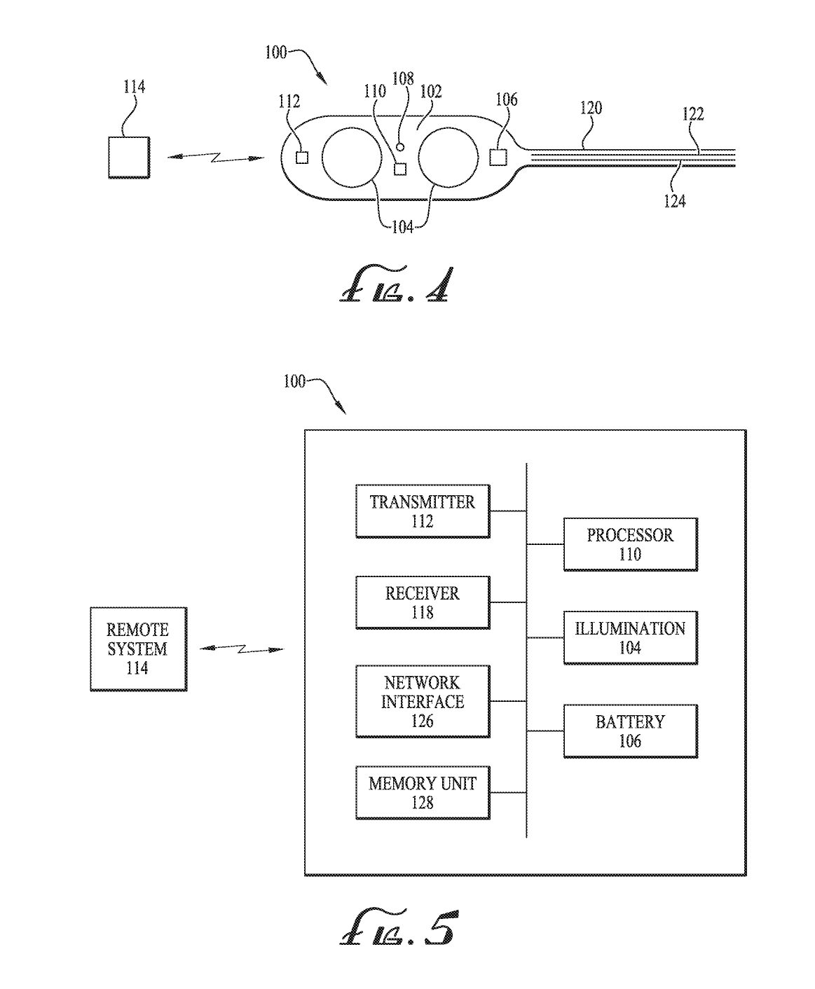

FIG. 4 illustrates another embodiment of a standalone stroke detection device 100 that may eliminate the need for a separate, external photodetector/sensor device 30. The following proceeds with a description of components and features of the stroke detection 100. It should be understood that the stroke detection device 100 may share many of the same or substantially similar features as the stroke detection device 50. For simplicity, the following description may not provide detail of some of these components to avoid obscuring more pertinent features of the stroke detection device 100, with the understanding that the components may operate in the same or substantially similar manner as described with respect to the stroke detection device 50.

With reference to FIG. 4, the stroke detection device 100 includes a fiberoptic port 102 connected to an end of a fiberoptic catheter 120. The fiberoptic port 102 includes an illumination/light source 104 operable to produce red or infrared light and a small battery 106 with a long life for powering the light source 104. In some embodiments, the device 100 may include a recharge port 108 in communication with the battery 106, where the battery 106 may be recharged through the skin such as by using intermittent electrical current delivered via a needle through the recharge port 108. In other embodiments, the battery 106 may instead be recharged by intermittent transcutaneous illumination of a minute photovoltaic cell housed on the surface of the device 100.

In an example operation, the light source 104 produces infrared light that is carried by the in-dwelling catheter fiberoptic channel (e.g., via the optical fiber 122). The light reflects off the red blood cells in the subcutaneous region surrounding the placement of the stroke detection device 100 within the jugular bulb in a similar fashion as described previously with respect to the stroke detection device 100. The reflected light is then detected by the device 100, such as via the second optical fiber 124.

In some embodiments, stroke detection device 100 may further include a processor 110 operable to analyze for evidence of decreased SⱼVO₂ levels to determine whether the patient 5 is experiencing an onset of an acute ischemic stroke. The device 100 may further include a transmitter 112 operable to wirelessly transmit (such as via Bluetooth™) the analysis results to a remote system 114, such as a bedside computer or other database. In other embodiments, the device 100 may omit the processor 110, and instead use the transmitter 112 to transmit the light data to an external computer or database for processing.

FIG. 5 is a schematic drawing illustrating an example arrangement of the internal electronics and components of the stroke detection device 100. With reference to FIG. 5, the device 100 includes a processor 110, which may be any of various suitable commercially available processors or other logic machine capable of executing instructions. In some embodiments, suitable dual microprocessors or other multi-processor architectures may also be employed as the processor 110.

The device 100 includes a network interface 126 to facilitate communication with one or more other devices, such as a remote system 114, which may be a server, a mobile device or phone, a computer, or any other suitable device. The network interface 126 may facilitate wireless communication with other devices over a short distance (e.g., Bluetooth™). Preferably, the device 100 uses a wireless connection, which may use low or high powered electromagnetic waves to transmit data using any wireless protocol, such as Bluetooth™, IEEE 802.11b (or other WiFi standards), infrared data association (IrDa), and radio frequency identification (RFID).

The device 100 further includes a transmitter 112 operable for transmitting data from the device 100 to the remote system 114 or to any other suitable device. For example, the transmitter 112 may transmit the reflected light data for external spectrophotometric analysis by the remote system 114, or may instead transfer the spectrophotometric analysis results completed internally by the stroke detection device 100. The device 100 may further include a receiver 118 operable for receiving data or instructions, such as for controlling the illumination sources 104, from the remote system 114 or any other paired device, and communicating the received data to the processor 110 for execution.

The device 100 further includes a memory unit 128, which may be implemented using one or more suitable memory devices, such as RAM and ROM. In one embodiment, any number of program modules may be stored in the memory unit 128, including an operating system, one or more application programs, patient data, storage files, device settings, and/or any other suitable modules for operation of the device 100. For example, the memory unit 128 may store historical patient data relating to SⱼVO₂ levels for the individual patient. After each testing protocol, the memory unit 128 may be updated with the test results to chart the progress of the SⱼVO₂ levels for the specific patient to more accurately assess the risk of an ischemic stroke.

The above-described components of the device 100, including the processor 110, the network interface 126, the transmitter 112, the receiver 118, the memory 128, and the battery 106, may be interconnected via a bus 116. It should be understood that while a bus-based architecture is illustrated in FIG. 5, other types of architectures are also suitable. In addition, in some embodiments, one or more components may be directly coupled to one another or combined as a single unit. For example, the transmitter 112 and receiver 118 may be combined into a single transceiver unit (not shown) to save space, provide an efficient component arrangement within the device 100, and reduce circuitry requirements.

In addition, while the illustrated embodiment depicts one possible configuration for the device 100, it should be recognized that a wide variety of hardware and software configurations may be provided without departing from the principles of the described embodiments. For example, other versions of the device 100 may have fewer than all of these components or may contain additional components.

It is intended that subject matter disclosed in any one portion herein can be combined with the subject matter of one or more other portions herein as long as such combinations are not mutually exclusive or inoperable. In addition, many variations, enhancements and modifications of the stroke detection device concepts described herein are possible.

The terms and descriptions used above are set forth by way of illustration only and are not meant as limitations. Those skilled in the art will recognize that many variations can be made to the details of the above-described embodiments without departing from the underlying principles of the invention.

1. An implantable stroke detection device comprising:
a catheter including a first optical fiber and a second optical fiber each extending along at least a portion of the catheter, the catheter insertable into a subcutaneous region of a patient's skin, the catheter configured to direct infrared light along the first optical fiber to illuminate the subcutaneous region, and to further obtain reflected light data via the second optical fiber based on the reflected infrared light from cells present in the subcutaneous region;
a fiberoptic port connected to one end of the catheter; and
a sensor in communication with the fiberoptic port, the sensor operable to receive the reflected light data.

2. The implantable stroke detection device of claim 1, further comprising a processor in operable communication with the sensor, the processor operable to analyze the reflected light data and determine SⱼVO₂ levels based on the reflected light data.

3. The implantable stroke detection device of claim 2, further comprising an illumination source operable to produce the infrared light.

4. The implantable stroke detection device of claim 3, further comprising a battery unit carried by the fiberoptic port, the battery unit operable to power the illumination source.

5. The implantable stroke detection device of claim 4, further comprising a recharge port in operable communication with the battery unit, the recharge port operable to recharge the battery unit.

6. The implantable stroke detection device of claim 1, further comprising a transmitter in operative communication with the processor and in wireless communication with a remote server, the transmitter configured to transmit to the remote server the reflected light data obtained by the catheter.

7. The implantable stroke detection device of claim 1, wherein the fiberoptic port further comprises at least one light receiver in communication with a transmitter, the light receiver configured to receive and direct infrared light to the catheter for illuminating the subcutaneous region.

8. The implantable stroke device of claim 1, wherein the sensor is external of the patient's skin and operable to receive the reflected light data through the patient's skin.

9. A stroke detection device comprising:
a catheter including a first optical fiber and a second optical fiber each extending along at least a portion of the catheter, the catheter insertable into a subcutaneous region of a patient's skin, the catheter configured to direct infrared light along the first optical fiber to illuminate the subcutaneous region, and to further obtain reflected light data via the second optical fiber based on the reflected infrared light from cells present in the subcutaneous region;
a fiberoptic port connected to one end of the catheter, the fiberoptic port positionable in the subcutaneous region of the patient's skin; and
an external sensor device in communication with the fiberoptic port through the patient's skin, the external sensor device operable to receive the reflected light data through the patient's skin.

10. The stroke detection device of claim 9, further comprising a processor in operable communication with the external sensor device, the processor operable to analyze the reflected light data and determine SⱼVO₂ levels based on the reflected light data.

11. The stroke detection device of claim 9, the external sensor device further comprising an illumination source operable to produce the infrared light, the illumination source directing the infrared light to the fiberoptic port through the patient's skin.

12. The stroke detection device of claim 9, the external sensor device further comprising a transmitter in operative communication with a remote server, the transmitter configured to transmit to the remote server the reflected light data obtained by the catheter.

13. The stroke detection device of claim 11, wherein the fiberoptic port further comprises at least one light receiver in communication with the external sensor device, the light receiver configured to receive and direct the infrared light to the catheter for illuminating the subcutaneous region.
